SEQUENCE LISTING (5 sequences)

SEQ ID NO: 1 - protein, 37 aa, Human immunodeficiency virus 1
MEPVNPRLEPWKHPGSQPKTACTNCYCAKCCFHCQVC

SEQ ID NO: 2 - protein, 24 aa, Human immunodeficiency virus 1
RAKRRQRRRPPQGSQTHQVSLSKQ

SEQ ID NO: 3 - protein, 18 aa, Human immunodeficiency virus 1
TACTNCYCAKCCFHCQVC

SEQ ID NO: 4 - protein, 9 aa, Human immunodeficiency virus 1
RAKRRQRRR

SEQ ID NO: 5 - protein, 72 aa, Human immunodeficiency virus 1
MEPVNPRLEPWKHPGSQPKTACTNCYCAKCCFHCQVCFITKALGISYGRAKRRQRRRPPQGSQTHQVSLSKQ

(19) United States
(12) Patent Application Publication
(10) Pub. No.: US 2025/0197453 A1
(43) Pub. Date: Jun. 19, 2025

(54) COMPOSITION COMPRISING TAT PEPTIDE VARIANT AS ACTIVE INGREDIENT FOR PREVENTING OR TREATING METABOLIC DISEASES

(71) Applicant: Man-Wook HUR, Seoul (KR)

(72) Inventors: Man-Wook HUR, Seoul (KR); Sun-Young PARK, Seoul (KR); Sujin PARK, Incheon (KR); Ji-Yang SONG, Daejeon (KR); Seohyun CHOI, Seoul (KR); Youngso YOON, Gimpo-si (KR)

(21) Appl. No.: 18/690,320

(22) PCT Filed: Sep. 8, 2022
(86) PCT No.: PCT/KR2022/013529

§ 371 Date: Oct. 17, 2024

(30) Foreign Application Priority Data
Sep. 10, 2021 (KR) 10-2021-0120788

(51) Int. Cl. C07K 14/005; A61K 38/00; A61P 3/04; A61P 3/10; C07K 7/06; C07K 7/08

(52) U.S. Cl. CPC C07K 14/005; A61P 3/04; A61P 3/10; C07K 7/06; C07K 7/08; A61K 38/00; C12N 2740/16022

(57) ABSTRACT

The present invention relates to a pharmaceutical composition and a functional food composition for preventing or treating metabolic disorders, which comprise, as an active ingredient, a TAT peptide variant in which internal residues of a certain length are replaced with hydrophobic aliphatic amino carboxylic acid or polyethylene glycol. The composition of the present invention has significantly increased water solubility and yield while retaining the fat-reducing effect of the natural TAT peptide. Thus, it may be useful as a composition for treating metabolic disorders that is easier to produce and has better biocompatibility.

### CROSS-REFERENCE TO RELATED APPLICATIONS

This application is a U.S. National Stage Entry of International Patent Application no. PCT/KR2022/013529, filed Sep. 8, 2022, which claims the benefit of priority of Korean Patent Application no. 10-2021-0120788, filed Sep. 10, 2021.

### REFERENCE TO AN ELECTRONIC SEQUENCE LISTING

This application contains a Sequence Listing which has been submitted electronically and is hereby incorporated by reference in its entirety. The Sequence Listing was created on Mar. 5, 2024, is named “24-0343-WO-US_SequenceListing_ST26” and is 5,584 bytes in size.

### TECHNICAL FIELD

The present invention relates to a composition for preventing or treating metabolic disorders comprising, as active ingredients, TAT peptide variants in which internal residues of a certain length are replaced with hydrophobic aliphatic amino carboxylic acid or polyethylene glycol.

### BACKGROUND ART

Obesity is a metabolic disorder caused by the imbalance between calorie intake and consumption, and is caused by the increased size (hypertrophy) or increased number (hyperplasia) of in vivo adipocytes. Obesity is not only the most common nutritional disorder in western society, but the prevalence of obesity in Korea has also recently tended to rapidly increase due to improved eating habits and westernization of lifestyles by economic development. According to OECD statistics in 2017, in Korea, the severely obese population is about 5.3% of the total population, which is not high compared to the OECD average obesity rate, but the obesity rate of male children and adolescents (26%, including overweight) is higher than the OECD average (25.6%), and it is predicted that the severely obese population will double by 2030 while the obesity rate tends to increase every year. In addition, socioeconomic losses due to obesity have doubled over the past 10 years to 9.2 trillion won in 2015, and are expected to further accelerate due to aging, etc.

Metabolic syndrome, which involves diabetes, hypertension, lipid metabolism abnormalities, and insulin resistance, etc., has emerged as a major group of diseases that threaten human health due to its high prevalence. In developed countries such as the United States and Europe, metabolic syndrome has been considered a serious health problem that threatens national competitiveness, and large amounts of human and material resources have been invested to solve this problem. Diseases belonging to the metabolic syndrome are a group of common diseases that increase the risk of mutual occurrence and are associated with multiple in vivo metabolic changes such as aging, stress, and decreased immune function.

Meanwhile, infection by HIV-1 (Human Immunodeficiency Virus-1) is the cause of AIDS (Acquired Immunodeficiency Syndrome), and metabolic abnormalities, weight loss, loss of appetite, and damage to body tissues are the major clinical symptoms of HIV infection. These changes, collectively referred to as rapid weight loss (wasting), are one of the major causes of morbidity and death in AIDS patients.

TAT is a small nuclear transcriptional activator protein encoded by HIV-1, and the amino acid sequence thereof is conserved in all primate lentiviruses (Myers et al. 1996). TAT is one of the most important regulators of HIV-1 transcription and replication, and is known to be responsible for several intracellular biological regulatory functions such as T-lymphocyte activation, apoptosis, and regulation of gene expression.

Throughout the specification, a number of publications and patent documents are referred to and cited. The disclosure of the cited publications and patent documents is incorporated herein by reference in its entirety to more clearly describe the state of the related art and the present invention.

### DISCLOSURE

### Technical Problem

The present inventors have made extensive efforts to develop an efficient therapeutic composition that has excellent therapeutic activity against metabolic disorders, including obesity, diabetes, dyslipidemia, fatty liver, and insulin resistance syndrome, and at the same time, has fewer side effects even when administered for a long period of time, and is easy to synthesize. As a result, the present inventors have identified the CDK9 binding domain and the Cyclin T1 binding domain, which play a key role in fat reduction, in the HIV-1-derived TAT peptide (72 a.a.) with anti-obesity activity, and have found that, when 11 amino acid residues between these domains are replaced with hydrophobic aliphatic amino carboxylic acid or polyethylene glycol while these domains are conserved, the water solubility and yield of the TAT peptide are significantly increased while the anti-obesity activity of the TAT peptide is retained, thereby completing the present invention.

Therefore, an object of the present invention is to provide a pharmaceutical composition or functional food composition for preventing or treating metabolic disorders.

Other objects and advantages of the present invention will become more apparent from the following detailed description of the invention, the appended claims and the accompanying drawings.

### Technical Solution

In accordance with one aspect of the present invention, the present invention provides a compound represented by following Formula 1 or a pharmaceutically acceptable salt thereof as an active ingredient:

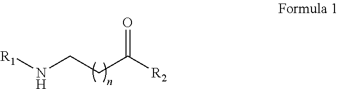

wherein R₁ is a polypeptide having the amino acid sequence of SEQ ID NO: 1 or a partial fragment of the C-terminal region thereof, R₂ is a polypeptide having the amino acid sequence of SEQ ID NO: 2 or a partial fragment of the N-terminal region thereof, and n is an integer of 3 to 7.

Specifically, the compound of Formula 1 may be synthesized by forming a peptide bond between the C-terminal amino acid of the R₁ polypeptide and the amine of the following aliphatic amino carboxylic acid and forming a peptide bond between the N-terminal amino acid of the R₂ polypeptide and the carboxylic acid of the following aliphatic amino carboxylic acid.

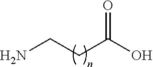

Accordingly, in Formula 1 above, —OH is removed from the carboxylic acid of the C-terminal amino acid of the R₁ polypeptide, and one hydrogen is removed from the amine group of the N-terminal amino acid of the R₂ polypeptide.

According to one embodiment of the present invention, the aliphatic amino carboxylic acid located between polypeptides R₁ and R₂ and connecting these two polypeptides with each other may be a linear or branched aliphatic amino carboxylic acid as shown in Formula 1 above.

The present inventors have made extensive efforts to develop an efficient therapeutic composition that has excellent therapeutic activity against metabolic disorders, including obesity, diabetes, dyslipidemia, fatty liver, and insulin resistance syndrome, and at the same time, has fewer side effects even when administered for a long period of time, and is easy to synthesize. As a result, the present inventors have succeeded in mapping the 20-57 a.a. region, which is an important region for fat reduction, in the HIV-1-derived TAT peptide (72 a.a.) with anti-obesity activity. This TAT fragment is composed of three domains (cysteine-zinc finger domain, core domain, and ARG-rich basic PTD domain) and exhibits the ability to inhibit adipocyte differentiation of 3T3-L1 preadipocytes and fat synthesis in adipocytes. Interestingly, as a result of analyzing the protein interaction site of the complex of TAT and CDK9-Cyclin T1, the present inventors have identified that the binding between TAT and Cyclin T1 is achieved through the TAT fragment (a.a. 20-57) mapped as an important region for fat reduction as described above, and that the alpha-helical peptide of each protein is formed by hydrophobic protein-protein interactions. Thereby, the present inventors have found that, when 11 amino acid residues forming the core domain are replaced with an aliphatic amino carboxylic acid of a certain length, the water solubility and yield of the TAT peptide are significantly increased while retaining the anti-obesity activity of the natural TAT peptide, making it easy to produce the TAT peptide and it possible to use the TAT peptide as an efficient therapeutic composition with excellent biocompatibility.

According to the present invention, the amino acid sequence of SEQ ID NO: 1 consists of residues 1 to 37 of the TAT peptide (72 a.a.), and the amino acid sequence of SEQ ID NO: 2 consists of residues 49 to 72 of the TAT peptide. Therefore, Formula 1 above represents a TAT peptide variant in which 11 residues (38 to 48) between the basic PTD domain, which is the CDK9 binding domain, and the cysteine zinc finger domain, which is the Cyclin T1 binding domain, are replaced with an aliphatic amino carboxylic acid.

According to a specific embodiment of the present invention, n is an integer of 3 to 6, more specifically 4 or 5, most specifically 4.

According to a specific embodiment of the present invention, R₁ in Formula 1 above is a polypeptide having the amino acid sequence of SEQ ID NO: 1 or has 18 or more amino acid residues which are consecutive in the direction from the C-terminus to the N-terminus of SEQ ID NO: 1. More specifically, the fragment is a polypeptide having the amino acid sequence of SEQ ID NO: 3.

According to a specific embodiment of the present invention, R₂ in Formula 1 above is a polypeptide having the amino acid sequence of SEQ ID NO: 2 or has 9 or more amino acid residues that are consecutive in the direction from the N-terminus to the C-terminus of SEQ ID NO: 2. More specifically, the fragment has the amino acid sequence of SEQ ID NO: 4.

According to the present invention, the amino acid sequence of SEQ ID NO: 3 consists of residues 20 to 37 of the TAT peptide (72 a.a.), and the amino acid sequence of SEQ ID NO: 4 consists of residues 49 to 57 of the TAT peptide.

In accordance with another aspect of the present invention, the present invention provides a pharmaceutical composition for preventing or treating a metabolic disorder selected from the group consisting of obesity, diabetes, dyslipidemia, fatty liver, and insulin resistance syndrome, comprising the above-described compound of the present invention or a pharmaceutically acceptable salt thereof as an active ingredient.

In accordance with still another aspect of the present invention, the present invention provides a method for preventing or treating a metabolic disorder selected from the group consisting of obesity, diabetes, dyslipidemia, fatty liver, and insulin resistance syndrome, comprising a step of administering the above-described compound of the present invention or a pharmaceutically acceptable salt thereof to a subject.

In the present specification, the term “metabolic disorder” refers to a group of diseases defined by conceptualizing the phenomenon in which risk factors for various cardiovascular diseases and type 2 diabetes caused by metabolic abnormalities are clustered together, and the term is intended to encompass insulin resistance and a variety of complex and diverse metabolic abnormalities and clinical aspects related thereto.

In the present specification, the term “obesity” refers to a condition in which energy intake exceeds energy consumption over a long period of time and excess energy is stored as fat, and this level of adipose tissue in vivo becomes excessive. Generally, obesity is clinically defined when the body mass index (=weight (kg)/[height (m)]²) is 25 or more.

In the present specification, the term “diabetes” refers to a chronic disease characterized by relative or absolute deficiency of insulin, resulting in glucose intolerance. Diabetes, which is treated or prevented with the composition of the present invention, includes all types of diabetes, for example, type 1 diabetes, type 2 diabetes, and hereditary diabetes. Type 1 diabetes is insulin-dependent diabetes, which is mainly caused by the destruction of β-cells. Type 2 diabetes is non-insulin-dependent diabetes, which is caused by insufficient insulin secretion after meals or by insulin resistance.

In the present specification, the term “dyslipidemia” refers to a pathologic condition in which the fat concentration in the blood is outside the normal range. Examples of dyslipidemia include hypercholesterolemia, hypertriglyceridemia, hypo-HDL-cholesterolemia, and hyper-LDL-cholesterolemia, as well as all abnormal lipid conditions caused by abnormal lipoprotein metabolism.

In the present specification, the term “fatty liver” refers to a condition in which excess lipid accumulates in liver cells due to a disorder of fat metabolism in the liver. Fatty liver is a cause of various diseases such as angina, myocardial infarction, stroke, arteriosclerosis, fatty liver, and pancreatitis.

In the present specification, the term “insulin resistance” refers to a condition in which cells cannot effectively burn glucose due to a decrease in the function of insulin that lowers blood glucose levels. If insulin resistance is high, the human body produces too much insulin, which can lead to hypertension or dyslipidemia as well as heart disease and diabetes. In particular, in type 2 diabetes, the increase in insulin is unrecognized in muscle and fat tissue, and thus insulin action does not occur. The term “insulin resistance syndrome” is a generic term for diseases caused by insulin resistance, and means diseases characterized by cell resistance against insulin action, hyperinsulinemia, increased very-low-density lipoprotein (VLDL) and triglycerides, decreased high-density-lipoprotein (HDL), and hypertension, and the insulin resistance syndrome is usually considered as a risk factor for cardiovascular disease and type 2 diabetes (Reaven G M, Diabetes, 37: 1595-607, (1988)).

In the present specification, the term “pharmaceutically acceptable salt” includes a salt derived from a pharmaceutically acceptable inorganic acid, organic acid, or base. Examples of suitable acids include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, trifluroacetic acid, citric acid, methanesulfonic acid, formic acid, benzoic acid, malonic acid, naphthalene-2-sulfonic acid, benzenesulfonic acid, and the like. Salts derived from suitable bases include salts of alkali metals such as sodium, alkaline earth metals such as magnesium, ammonium, and the like.

According to a specific embodiment of the present invention, dyslipidemia to be prevented or treated with the composition of the present invention is hyperlipidemia.

The term “hyperlipidemia” as used in the present specification refers to a disease that occurs when metabolism of fats such as triglycerides and cholesterol is poor and lipid levels in the blood are maintained at high levels. More specifically, hyperlipidemia refers to a state in which lipid components such as triglycerides, LDL cholesterol, phospholipids, and free fatty acids in the blood are increased. Hyperlipidemia includes hypercholesterolemia or hypertriglyceridemia, which occurs with a high incidence.

According to a specific embodiment of the present invention, fatty liver to be prevented or treated with the composition of the present invention is non-alcoholic fatty liver.

In the present specification, the term “non-alcoholic fatty liver (NAFL)” refers to a disease in which excess lipid accumulates in liver cells regardless of the uptake of alcohol, and includes simple fatty liver (steatosis) and non-alcoholic steatohepatitis (NASH). Simple fatty liver has a good clinical prognosis, but NASH, accompanied by inflammation or fibrosis, is a progressive liver disease and is recognized as a precursor disease that causes cirrhosis or liver cancer. Obesity and insulin resistance are representative risk factors for non-alcoholic fatty liver disease. Risk factors for the progression of liver fibrosis include obesity (BMI>30), the ratio of liver function parameters detected in serum (AST/ALT>1), and diabetes. Particularly, when hepatitis C carriers have non-alcoholic fatty liver, the non-alcoholic fatty liver can progress to liver cancer. 69 to 100% of patients with non-alcoholic fatty liver disease are obese, and 20 to 40% of obese patients have non-alcoholic fatty liver disease. In particular, 10 to 77% of obese children in Europe, the United States, and Asia have non-alcoholic fatty liver lesions, because obesity is the most important risk factor for non-alcoholic liver disease.

In the present specification, the term “prevention” means inhibiting the occurrence of a disorder or a disease in a subject who has never been diagnosed with the disorder or disease, but is likely to suffer from such disorder or disease.

In the present specification, the term “treatment” means (a) inhibiting the progress of a disorder, disease or symptom; (b) alleviating the disorder, disease or symptom; or (c) eliminating the disorder, disease or symptom. When the composition of the present invention is administered to a subject, it functions to reduce the weight of adipose tissue, increase insulin and glucose sensitivity, and significantly reduce the expression of proteins involved in adipogenesis and cholesterol synthesis, thereby suppressing the development of metabolic disorder symptoms due to excessive accumulation of fat, or eliminating or alleviating the metabolic disorder symptoms. Thus, the composition of the present invention may serve as a therapeutic composition for the disorder alone, or may be administered in combination with other pharmacological ingredients and applied as a therapeutic aid for the disorder. Accordingly, as used in the present specification, the term “treatment” or “therapeutic agent” encompasses “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition containing a pharmacological ingredient sufficient to provide a therapeutic or prophylactic effect to a subject to whom the pharmaceutical composition of the present invention is to be administered. Accordingly, the term “therapeutically effective amount” encompasses a “prophylactically effective amount”.

In the present specification, the term “subject” includes, without limitation, humans, mice, rats, guinea pigs, dogs, cats, horses, cows, pigs, monkeys, chimpanzees, baboons or rhesus monkeys. Specifically, the subject of the present invention is a human.

When the composition of the present invention is prepared as a pharmaceutical composition, the pharmaceutical composition of the present invention comprises a pharmaceutically acceptable carrier.

Examples of the pharmaceutically acceptable carrier that is comprised in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methylcellulose, hydroxybenzoate, propyl hydroxybenzoate, talc, magnesium stearate, and mineral oil, which are commonly used in the formulation. The pharmaceutical composition of the present invention may further comprise a lubricant, a wetting agent, a sweetening agent, a flavoring agent, an emulsifying agent, a suspending agent, a preservative, and the like, in addition to the above-described components. Suitable pharmaceutically acceptable carriers and agents are described in detail in Remington's Pharmaceutical Sciences (19^{th} ed., 1995).

The pharmaceutical composition of the present invention may be administered orally or parenterally. Specifically, it may be administered orally, intravenously, subcutaneously or intraperitoneally.

An appropriate dosage of the pharmaceutical composition of the present invention may vary depending on various factors such as formulation method, administration mode, patient's age, weight, sex, pathological condition, diet, administration time, administration route, excretion rate, and reaction sensitivity. A preferred dosage of the pharmaceutical composition of the present invention is within the range of 0.001 to 100 mg/kg for an adult.

The pharmaceutical composition of the present invention may be prepared in a unit dose form or prepared to be contained in a multi-dose container by formulating with a pharmaceutically acceptable carrier and/or excipient, according to a method that may be easily carried out by a person skilled in the art. Here, the formulation of the pharmaceutical composition may be a solution, suspension, syrup or emulsion of the pharmaceutical composition in oil or aqueous medium, or an extract, powder, granule, tablet or capsule, and may further comprise a dispersing agent or a stabilizer.

In accordance with yet another aspect of the present invention, the present invention provides a food composition for ameliorating or alleviating a metabolic disorder selected from the group consisting of obesity, diabetes, dyslipidemia, fatty liver, and insulin resistance syndrome, comprising the above-described compound of the present invention or a food-acceptable salt thereof as an active ingredient:

In the present specification, the term “food-acceptable salt” refers to salt in a form that may be used in a food composition, among salts composed of cations and anions bound together by electrostatic attraction, and specific examples thereof include the above-described examples of the “pharmaceutically acceptable salt”.

When the composition of the present invention is prepared as a food composition, it may comprise not only the compound of the present invention as an active ingredient, but also carbohydrates, seasonings, and flavoring agents, which are commonly added in food preparation. Examples of the carbohydrates include, but are not limited to, monosaccharides such as glucose and fructose, disaccharides such as maltose and sucrose, polysaccharides such as dextrins and cyclodextrins, and sugar alcohols such as xylitol, sorbitol, and erythritol. Examples of the flavoring agents include natural flavoring agents [thaumatin, stevia extract (e.g., rebaudioside A, glycyrrhizin, etc.)] and synthetic flavoring agents (saccharin, aspartame, etc.). For example, when the food composition of the present invention is prepared as a drink, it may further comprise, in addition to a pine bark extract as an active ingredient of the present invention, citric acid, liquid fructose, sugar, glucose, acetic acid, malic acid, fruit juice, eucommia extract, jujube extract, licorice extract, or the like.

According to still yet another aspect of the present invention, the present invention provides a compound represented by following Formula 2 or a pharmaceutically acceptable salt thereof:

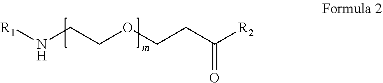

wherein R₁ is a polypeptide having the amino acid sequence of SEQ ID NO: 1 or a partial fragment of the C-terminal region thereof, R₂ is a polypeptide having the amino acid sequence of SEQ ID NO: 2 or a partial fragment of the N-terminal region thereof, and m is an integer of 4 to 7.

Specifically, R₁ and R₂ in Formula 2 above are the same as R₁ and R₂ defined in Formula 1 above.

Specifically, the compound of Formula 2 may be synthesized by forming a peptide bond between the C-terminal amino acid of the R₁ polypeptide and the amine of the following polyethylene glycol derivative and forming a peptide bond between the N-terminal amino acid of the R₂ polypeptide and the carboxylic acid of the following polyethylene glycol derivative:

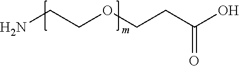

Accordingly, in Formula 2 above, —OH is removed from the carboxylic acid of the C-terminal amino acid of the R₁ polypeptide, and one hydrogen is removed from the amine group of the N-terminal amino acid of the R₂ polypeptide.

The present inventors have found that, in addition to the compound of Formula 1 in which the core domain composed of 11 amino acid residues (residues 38 to 48), among the domains of the TAT peptide (72 a.a.) with anti-obesity activity that binds to Cyclin T1 of the CDK9-Cyclin T1 complex, is replaced with one hydrophobic aliphatic amino carboxylic acid, the compound of Formula 2, obtained by substituting the compound of Formula 1 with polyethylene glycol of an appropriate length, may efficiently mimic the three-dimensional characteristics of the TAT peptide and the spatial relationship between the domains of the TAT fragment (a.a. 20-57) that binds to Cyclin T1, and at the same time, may exhibit the effect of increasing in vivo stability and availability due to PEGylation.

According to a specific embodiment of the present invention, m is 4 to 7, more preferably 6 to 8, and most preferably 7.

### Advantageous Effects

The features and advantages of the present invention are summarized as follows:

(a) The present invention provides a pharmaceutical composition and a functional food composition for prevention or treatment of metabolic disorders, which comprise, as an active ingredient, a TAT peptide variant in which internal residues of a certain length are replaced with hydrophobic aliphatic amino carboxylic acid or polyethylene glycol.

(b) The composition of the present invention has significantly increased water solubility and yield while retaining the fat-reducing effect of the natural TAT peptide. Thus, it may be useful as a composition for treatment of metabolic disorders that is easier to produce and has better biocompatibility.

### BRIEF DESCRIPTION OF DRAWINGS

FIG. 1 shows the crystal structure of the TAT-Cyclin T1-CDK9 complex, analyzed by X-ray diffraction. The structure comprising the cysteine zinc finger domain and core domain of TAT binds to Cyclin T1 via hydrophobic protein-protein interactions. The Arg-rich basic PTD domain does not appear in the structure due to its mobility.

FIG. 2 shows the structures of novel polypeptides (X-FAT5 (TAT-38 Apn) to X-FAT8 (replaced by TAT38 Aoc)) newly designed based on domain function studies of the TAT protein. The core domain (polypeptide consisting of 11 amino acids) is replaced with each of 5-amino-pentanoic acid, 6-amino-hexanoic acid, 7-amino-heptanoic acid, and 8-amino-octanoic acid.

FIG. 3 shows that X-FAT8 (FIG. 3A), X-FAT5 (FIG. 3B), and X-FAT5 to X-FAT8 (FIGS. 3C and 3D) inhibit differentiation of preadipocytes (3T3-L1) and inhibit adipogenesis, and X-FAT8 inhibits fatty acid synthesis and accumulation in human hepatocytes (HepG2) (FIG. 3E).

FIG. 4 shows Western blot analysis results indicating that the expression of proteins involved in the regulation of adipose tissue differentiation, adipogenesis, and cholesterol synthesis is decreased by administration of X-FAT8 (FIG. 4A) and X-FAT6 (FIG. 4B), and shows the mechanism of action by which the TAT peptide variant of the present invention inhibits adipogenesis (FIG. 4C).

FIG. 5 shows the significant weight loss in diet-induced obese (DIO) mouse model by treatment with X-FAT8 (FIG. 5A), X-FAT6 (FIG. 5B), and X-FAT7 (FIG. 5C). In the experiment shown in FIG. 5B, the weight loss effect of X-FAT6 was measured under the condition of feeding HFD. As a result of changing the diet to a normal diet without drug treatment to remove the effects of the drug remaining in the body and monitoring the weight change, diet change to the normal diet caused a rapid weight loss in the control and test groups.

FIG. 6 shows the change in food intake after the treatment of DIO mouse model with X-FAT8.

FIG. 7 shows the status of abdominal fat and changes in epididymal white adipose tissue (eWAT), liver tissue, and brown adipose tissue (BAT) after intraperitoneal administration of X-FAT8 in DIO mouse model.

FIGS. 8A and 8B show the results of performing GTT (glucose tolerance test) (FIG. 8A) and ITT (insulin tolerance test) (FIG. 8B) to examine changes in glucose homeostasis after intraperitoneal administration of X-FAT8 in DIO mouse model. GTT and ITT were performed in the same manner using X-FAT6 (FIG. 8C) and X-FAT7 (FIG. 8D).

FIG. 9 shows that administration of X-FAT6 (FIG. 9A) and X-FAT7 (FIG. 9B) exhibits the effect of reducing the size of adipocytes and dramatically ameliorating liver steatosis in DIO mouse model.

FIG. 10 shows the change in blood lipid concentration by administration of X-FAT6. As can be seen therein, TCHO (total cholesterol) and TG (triglyceride) levels decreased, and GPT and GOT levels also decreased, resulting in a decrease in liver fat, reducing liver injury and improving liver function.

FIG. 11 shows the results of analyzing the effects of PEGNn-TAT variants on differentiation and adipogenesis of 3T3-L1 cells. On 4 days after the initiation of differentiation, PEGn-TAT variants showed differentiation inhibitory ability and also reduced intracellular lipid accumulation. In particular, the inhibitory ability of the PEG7-TAT variant was shown to be very high (FIG. 11A). On 6 days after the induction of differentiation, the inhibitory ability of the PEG7-TAT variant was still significant (FIG. 11B).

FIG. 12 shows the results of analyzing the effects of PEGNn-TAT variants on differentiation and adipogenesis of 3T3-L1 cells. On 4 days (FIG. 12A) and 6 days (FIG. 12B) after the initiation of differentiation, PEGn-TAT variants showed differentiation inhibitory ability and also reduced intracellular lipid accumulation. In particular, the inhibitory ability of the PEG7-TAT variant was shown to be very high

FIG. 13 shows the results of analyzing the effects of PEGNn-TAT variants on differentiation and adipogenesis of 3T3-L1 cells by Oil-O-Red staining. On 8 days after the initiation of differentiation, the PEG7-TAT variant caused strong inhibition of differentiation and inhibition of intracellular lipid accumulation.

FIG. 14 shows the results of analyzing the effects of PEGNn-TAT variants on the inhibition of fatty acid synthesis in hepatocytes (HepG2) by Oil-O-Red staining. The control group was treated with oleic acid alone, and the experimental group was treated with oleic acid plus TAT-38, X-Fat8, or PEGn-TAT (n=7 to 11).

FIG. 15 shows the results of analyzing, by Oil-O-Red staining, the adipogenesis inhibitory effects of PEGn-TAT variants in HepG2 hepatocytes upon treatment with oleic acid. All PEGylated PEGn-TAT variants [PEGn-TAT (n=7 to 11)] used exhibited better inhibitory effects than TAT-38 and X-FAT8.

FIG. 16 shows the results of analyzing the structures of X-FAT5 (FIG. 16A), X-FAT6 (FIG. 16B), X-FAT7 (FIG. 16C) and X-FAT8 (FIG. 16D) peptides of the present invention through the MS spectra thereof after synthesis of the peptides.

### MODE FOR INVENTION

Hereinafter, the present invention will be described in more detail with reference to examples. These examples are only for explaining the present invention in more detail, and it will be apparent to those skilled in the art that the scope of the present invention according to the subject matter of the present invention is not limited by these examples.

### EXAMPLES

### Example 1: X-FATn Peptide Variants

### Animal Model

To test the in vivo efficacy of the anti-obesity peptides of the present invention, DIO (diet-induced obese) model mice (male, C57BL/6J DIO 12-week old, JAX LAB, Central Laboratory Animal Inc.) were used and fed a 60% high-fat diet for DIO purposes. The experimental animals were divided into three groups and orally administered the anti-obesity peptides of the present invention.

### Screening of Novel Anti-Obesity Peptides

The present inventors identified in previous studies that the significant weight loss (slim disease) reaching 20 to 30% observed between 0.5 and 2 years after infection with HIV-1 virus (1-3) is attributable to lipodystrophy caused by the Tat peptide, which is one of the HIV-1 gene products, and discovered, through domain mapping studies of the TAT peptide, a TAT-38 region (TACTNCYCAKCCFH-FITKALGISYG-RAKRRARQRRR) with anti-obesity activity (U.S. Pat. No. 10,300,111) (4). The discovered TAT-38 peptide was found to reduce the weight of adipose tissue and increase insulin and glucose sensitivity.

In the present invention, the inventors tried to develop the improved peptides of TAT-38 that have better pharmacological properties than TAT-38 and are easy to synthesize. The present inventors noted that the expression of CDK9 and Cyclin T1 is increased in obesity, and that substrates of CDK9, such as C/EBPa and PPAR-gamma, are important factors in adipogenesis. In addition, it was found that, when TAT binds to the CDK9-Cyclin T1 complex, it induces a change in the structure of CDK9. Therefore, the present inventors have analyzed the structure of CDK9-Cyclin T1-TAT, and as a result, have fortunately found that the three domains of TAT-38 fit perfectly into the binding pocket of Cyclin T1. It is thought that TAT-38 binds to the binding site of Cyclin T1, induces a structural change in CDK9 through the formation of a CDK9-Cyclin T1-TAT complex, and inhibits the activation of transcription factors, such as C/EBPa and PPAR-gamma, which are important in differentiation and growth of adipocytes, thereby inhibiting adipogenesis, thus reducing the size of adipose tissue.

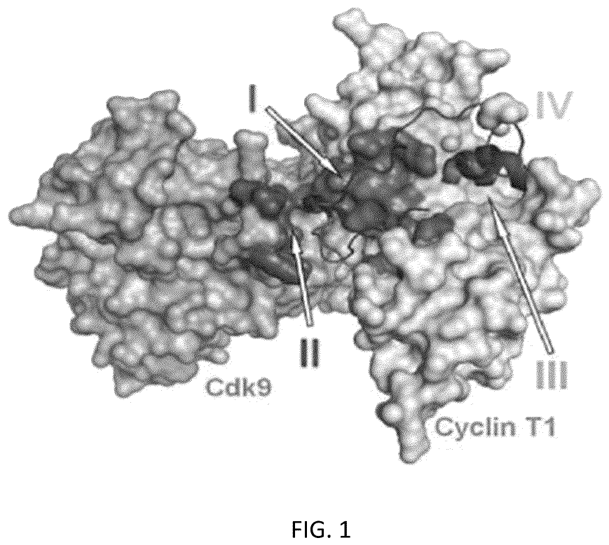

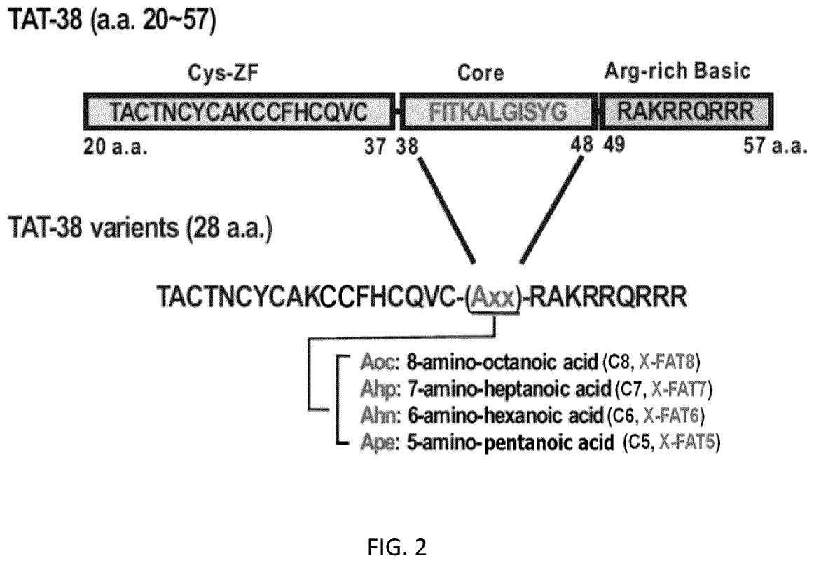

Furthermore, the present inventors analyzed crystalline structural data of a complex of the three proteins. As shown in FIG. 1, the cysteine zinc finger domain and core domain of TAT are bound to the alpha-helices of Cyclin T1 via hydrophobic protein-protein interactions. The Arg-rich basic PTD domain is not visible in the structure due to its mobility. All of the three domains are important in the inhibition of adipocyte differentiation and fatty acid synthesis, and in particular, the present inventors noted that the core domain is a polypeptide composed of 11 amino acids with mainly hydrophobic side-chains. The present inventors believed that this domain contributes to the low water solubility exhibited by TAT-38, and replaced this domain with a synthetic amino acid having one hydrophobic aliphatic linear or branched side-chain having a length similar to the longitudinal axis of the unique horn-shaped α-helix formed by 11 amino acids, thereby synthesizing TAT-38 analogues which have increased water solubility while having maintained or increased activity and whose synthetic efficiency can be increased by reducing the number of peptide binding reaction steps. Based on the fact that the length of the longitudinal axis of the core domain secondary structure is similar to the length of pentane or octane with 5 to 8 linearly linked carbon atoms, the present inventors designed four modified peptides in which 11 amino acids are removed and replaced with one 5-amino-pentanoic acid or 8-amino octanoic acid (FIG. 2). The present inventors named these modified TAT-38 peptides X-FAT5, X-FAT6, X-FAT7, and X-FAT8, respectively.

### Characterization of X-FAT8

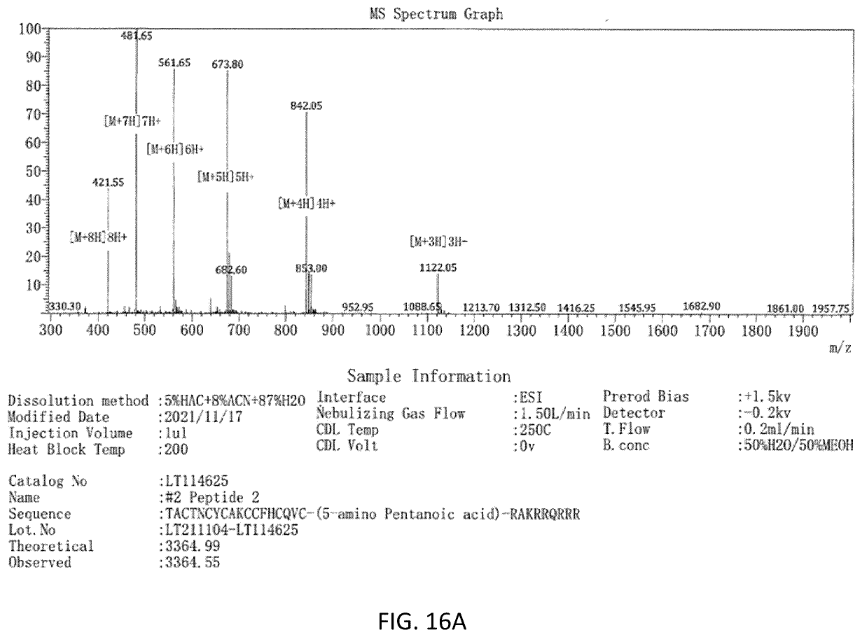

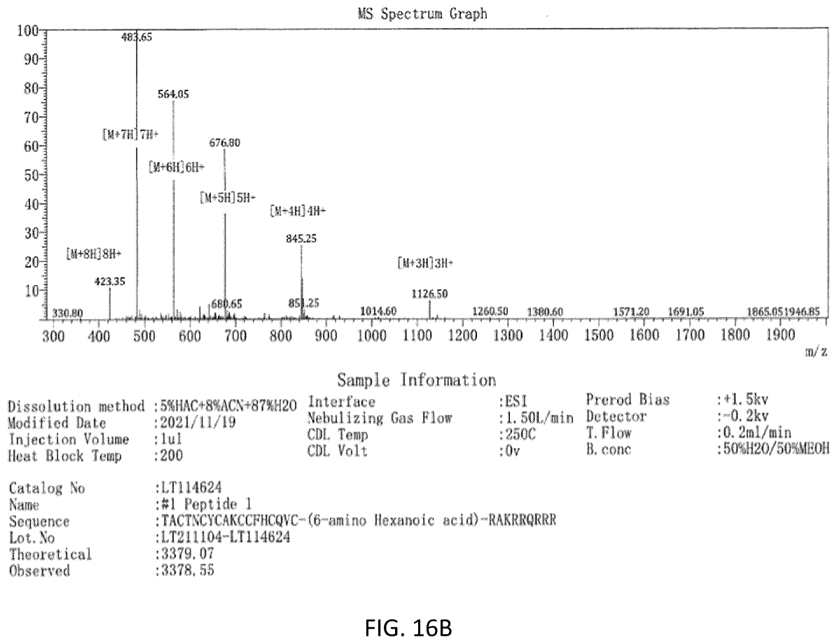

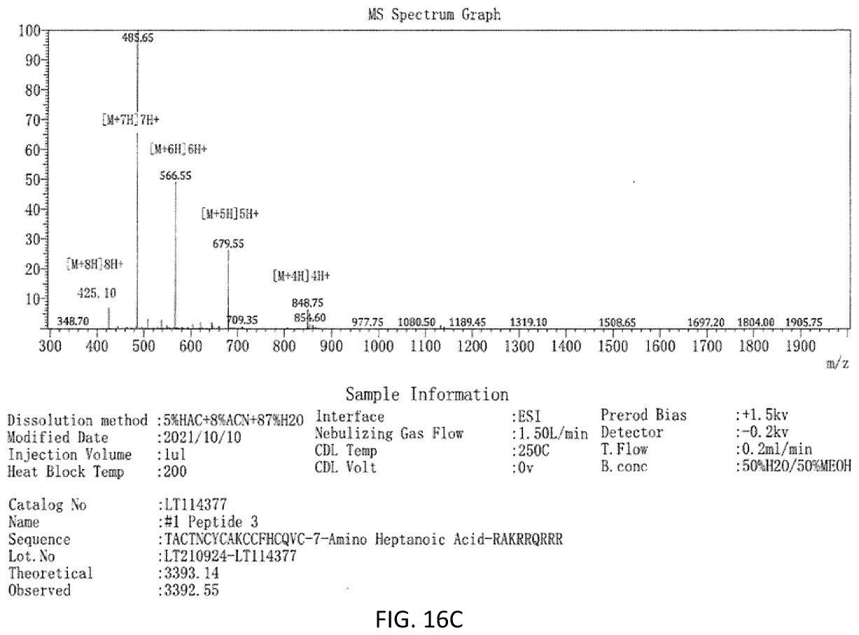

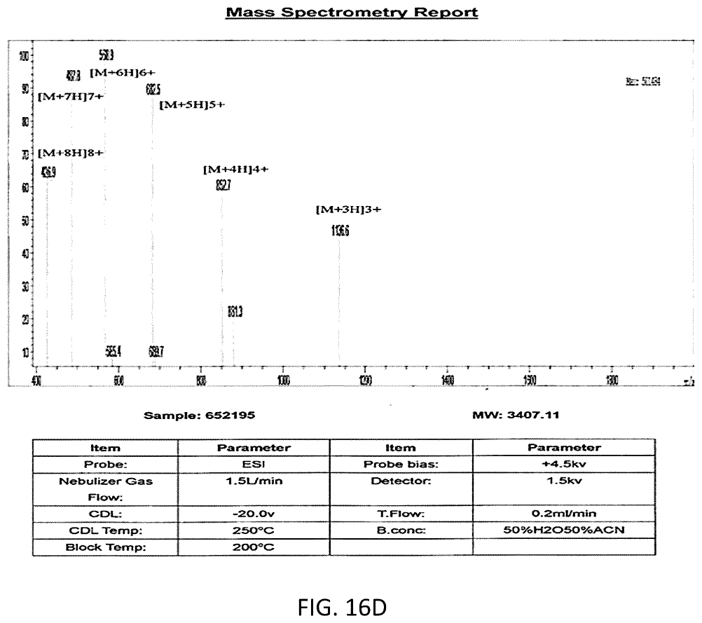

X-FAT5 to X-FAT8 peptides were synthesized on a small scale and the structures thereof were analyzed through mass spectrometry (MS) spectra thereof (FIG. 16). After verifying the pharmacological efficacy, each peptide was synthesized in a large amount (10 g) and dispensed (5 mg/vial, vacuum state) to prepare for the demand in experiments related to preclinical testing, etc. It was confirmed that each peptide showed a higher synthesis yield than TAT-38 and was freely soluble in water.

### Cell Experiment

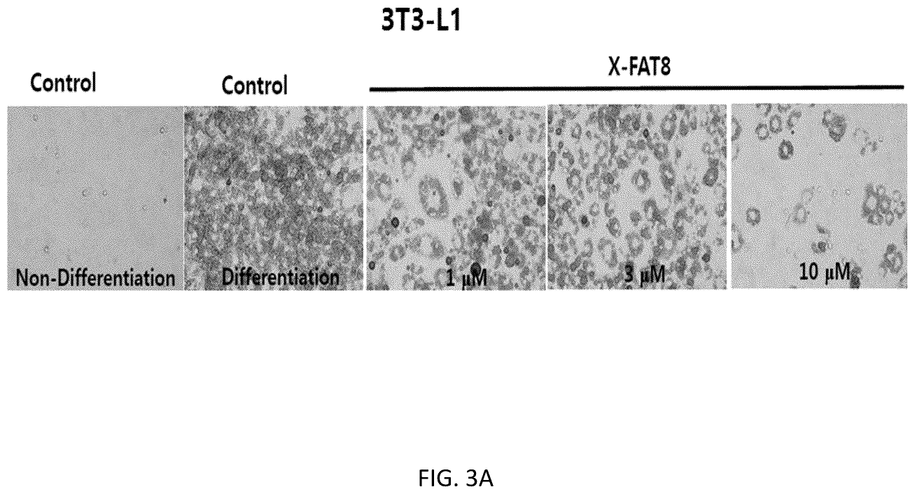

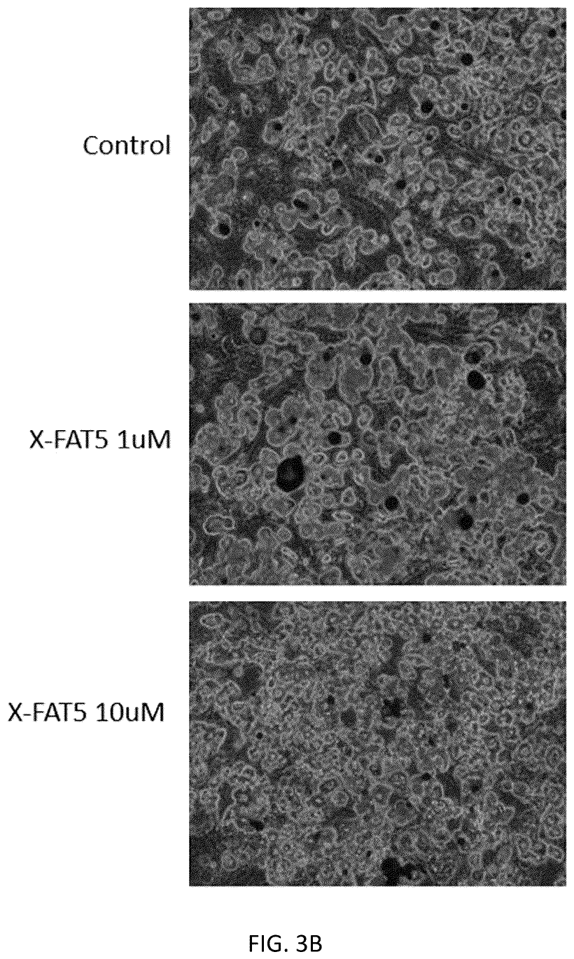

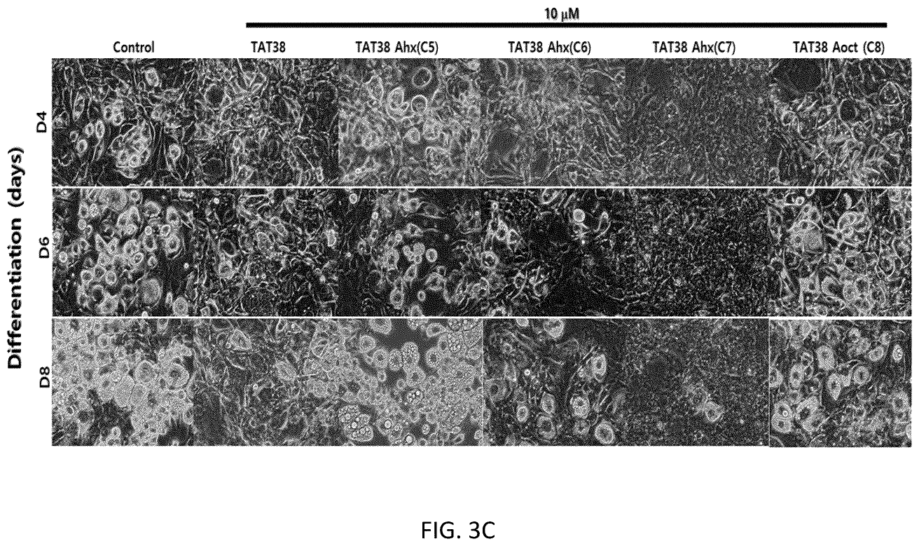

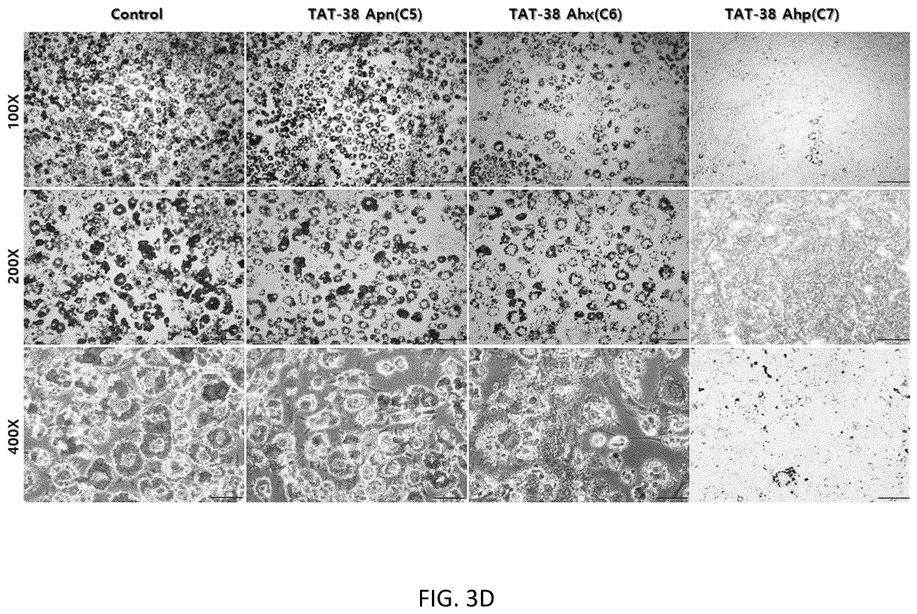

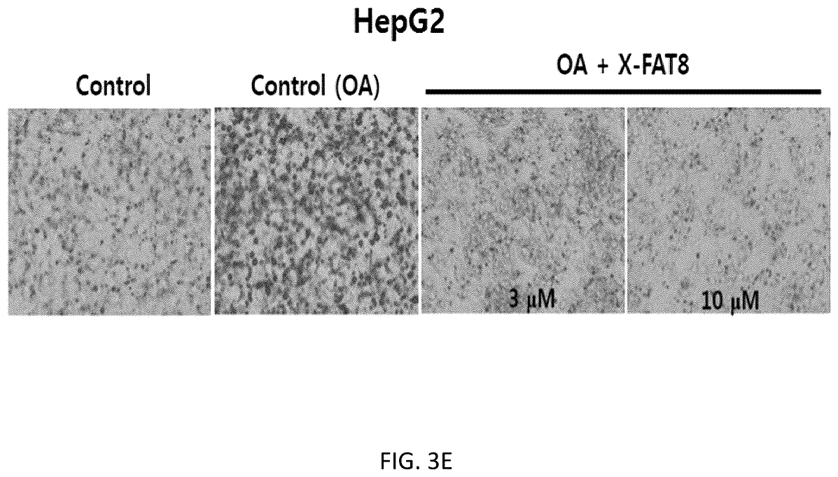

For culture of preadipocytes, DMEM (glucose 4.5 g/liter, GibcoBRL-Cat #11995-065) containing calf serum (Gibco-BRL-Cat #16170-078) dissolved at a final concentration of 10% was used. When the preadipocytes grew to about 70 to 80% of the culture flask area, the cells were detached from the flask by treatment with 0.05% trypsin-EDTA (GibcoBRL-Cat #15400-054) and diluted in medium, and the cell suspension was centrifuged at 1,200 rpm for 2 minutes. Then, the supernatant was removed and the cell pellet was resuspended in a fresh medium. The cells were counted and then dispensed into 60-mm round culture dishes at 5×10⁴ cells/culture dish. When the cells attached, the medium was replaced with 10% FBS/DMEM (Fetal Bovine Serum, GibcoBRL-Cat #10437-028; Dulbecco's Modified Eagle's Medium; DMEM/glucose 4.5 g/liter, GibcoBRL-Cat #11995-065), and the cells were cultured until they reached confluence. The cells were added to 1 ml of 10% FBS/DMEM supplemented with 1 μl of differentiation cocktail (3T3-L1 differentiation kit, cat #DIF001, Sigma) containing differentiation medium isobutylmethylxanthine (IBMX), insulin, dexamethasone, and rosiglitazone, and 1 μM, 3 μM, and 10 μM of X-FAT8 were further added to the medium, followed by 2 days of culture (day 0). The old medium was replaced with 10% FBS/DMEM supplemented with 1 μg/ml insulin, and 1 μM, 3 μM, and 10 μM of X-FAT8 were added to the medium, followed by 2 days of additional culture (day 2). It was observed that lipids accumulated in the cells in the control group after replacement with the medium containing insulin. Additionally, 1 μM, 3 μM and 10 μM of X-FAT8 were added to the replaced 10% FBS/DMEM, followed by 2 days, 4 days, 6 days, and 8 days of additional culture. Whether differentiation occurred was examined through microscopic observation and Oil Red O staining of the treated cells. In the control group, fully differentiated adipocytes were observed after 2 days, but in the experimental groups to which 1 μM, 3 μM, and 10 μM of X-FAT8 were added, the adipocyte differentiation and adipogenesis of 3T3-L1 cells were significantly inhibited in a manner dependent on the dose of X-FAT8 (FIG. 3A). In addition, as a result of performing Oil Red O staining in the same process using 1 μM and 10 μM of X-FAT5, it was confirmed that the adipocyte differentiation and adipogenesis of 3T3-L1 cells were also significantly inhibited (FIG. 3B).

In addition, as a result of comparing the effects of the modified peptides by performing the same experiment using TAT-38, X-FAT5, X-FAT6, X-FAT7 and X-FAT8 at a final concentration of 10 μM, it was confirmed that these peptides all significantly inhibited the adipocyte differentiation and adipogenesis of 3T3-L1 cells, but TAT-38 Ahp(C7) (X-FAT7) showed the strongest differentiation inhibitory ability, and then TAT-38 Ahn(C6) (X-FAT6) showed an adipogenesis inhibitory effect similar to that of TAT-38 (FIGS. 3C and 3D).

In addition, human HepG2 hepatocytes significantly increase intracellular lipid accumulation in the presence of oleic acid, and it was confirmed that lipid accumulation in HepG2 liver-derived cells was very effectively inhibited by administered X-FAT8 (FIG. 3E).

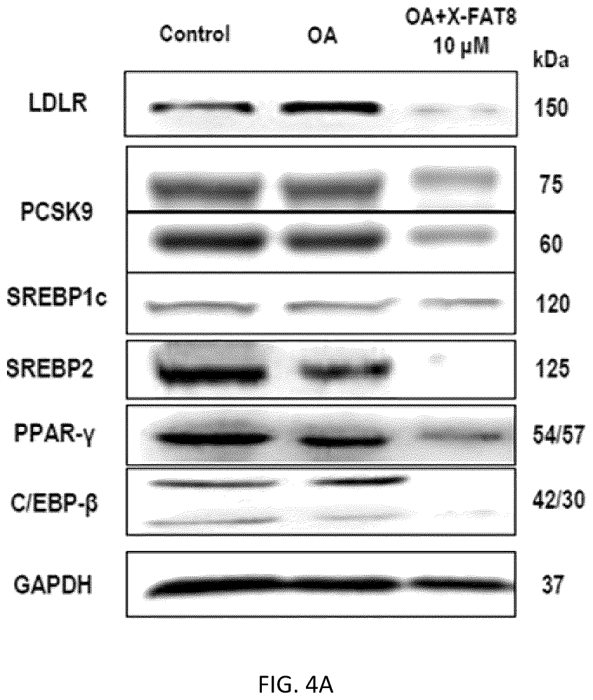

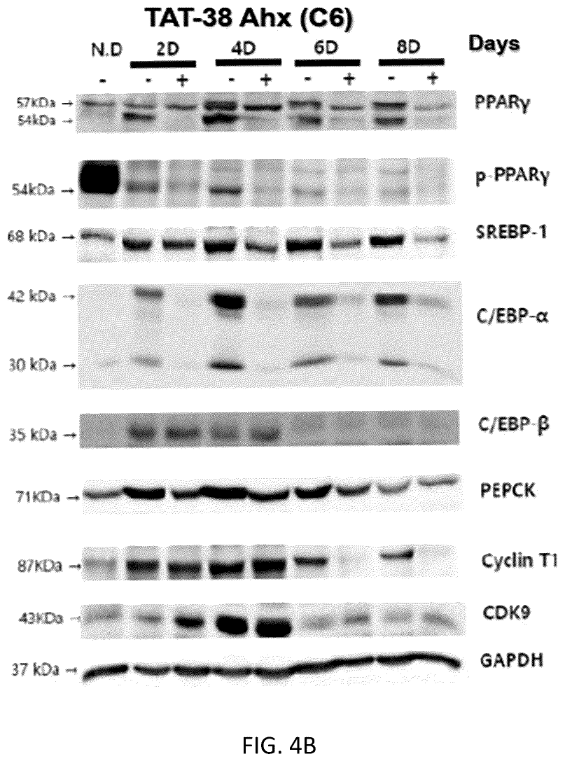

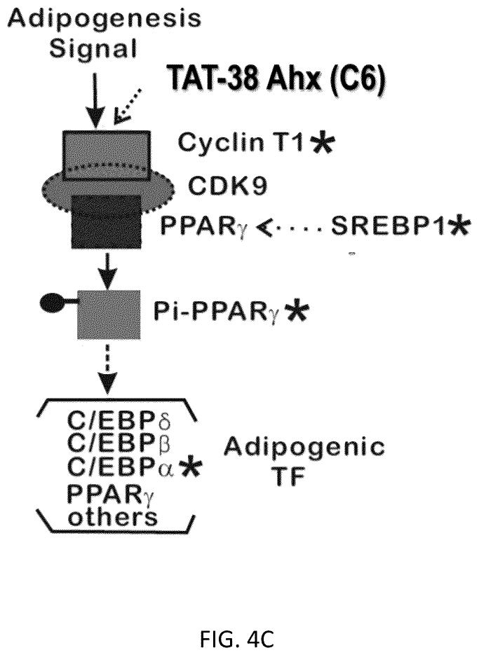

In addition, proteins which are changed by the treatment of HepG2 cells with X-FAT8 are presumed to be proteins involved in adipocyte differentiation, adipogenesis, cholesterol synthesis, etc., and thus the expression of LDLR, PCSK9, SREBP1c, SREBP2, PPAR-γ and C/EBP-β proteins was analyzed by Western blotting. HepG2 cells were treated with X-FAT8 (10 μM), and the cell extract (50 μg) was separated by 8% SDS-PAGE and transferred to the PVDF membrane, and then the expression of the proteins was examined using several antibodies (PCSK9, custom-made, Yonsei University College of Medicine; other antibodies produced by Santa Cruz; SC-8984; PPAR-γ, SC7273; GAPDH, SC-32233; C/EBPα (14 a.a.) SSC-61; SREBP-2, SC271616; PGC-1α(H300), SC-13067). As a result, it was found that the expression of these proteins was significantly reduced (FIG. 4A). Based on the action of X-FAT8 to reduce intracellular lipids, it was found that X-FAT8 can be applied as a useful therapeutic composition for various metabolic disorders caused by abnormal lipid metabolism. Meanwhile, changes in the expression of these lipid metabolism-related genes were likewise observed when treated with X-FAT6 (FIG. 4B).

### Experiment Using Obese Mouse Model

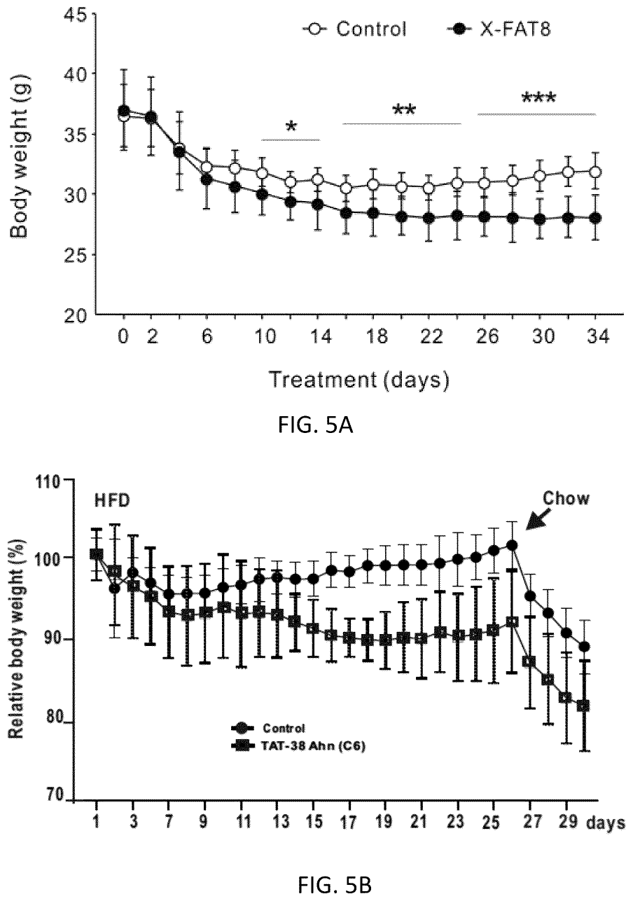

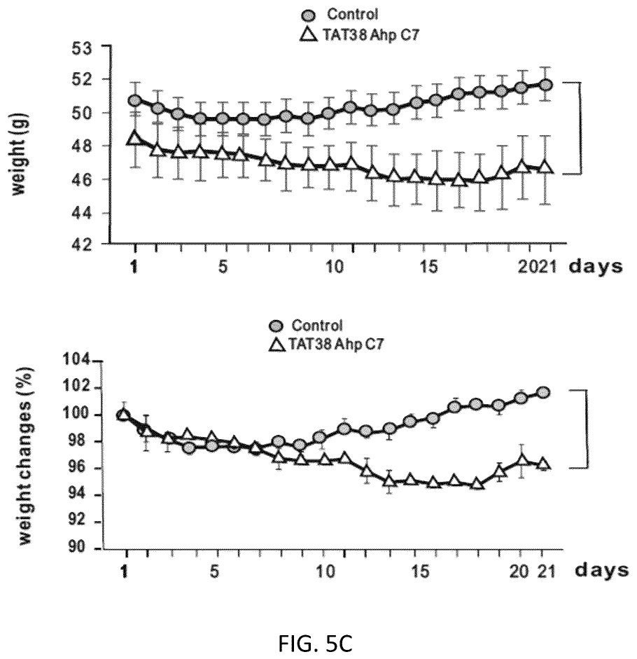

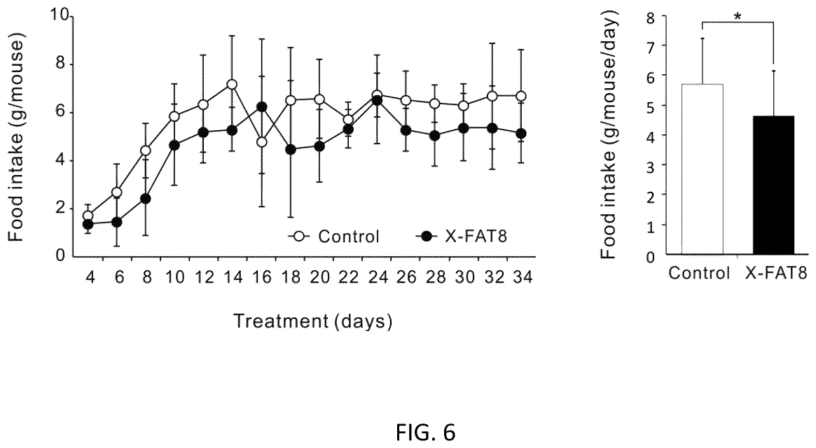

To test the in vivo efficacy of the anti-obesity peptides of the present invention, DIO (diet-induced obese) model mice (male, C57BL/6J DIO 12-week old, JAX LAB, Central Laboratory Animal Inc.) were used and fed a 60% high-fat diet for DIO purposes. The experimental animals were divided into two groups. The control group was administered a solvent (sterile distilled water) containing the sample dissolved therein, and the experimental group was orally administered an aqueous solution (150 μL) in which the anti-obesity peptide of the present invention was dissolved. As a result of administering X-FAT8 to the high-fat diet-induced obese (DIO) mouse model at a dose of 1 mg/ea once every two days for 34 days, a weight loss effect reaching 10 to 15% was shown (FIG. 5A), and as a result of orally administering X-FAT6 at a dose of 1 mg/mouse once daily for 27 days, the control group showed a 0.5 g increase (101.2%) compared to the initial body weight, whereas the experimental group showed a 3.7 g decrease (91.8%) compared to the initial body weight, indicating a relative body weight loss of 9.4% (FIG. 5B). In addition, as a result of orally administering X-FAT7 at a dose of 1 mg/mouse once every two days for 21 days, an about 5.5 g decrease (90%) compared to the initial body weight was shown, which corresponds to a relative body weight loss of 10% (FIG. 5C). However, no statistically significant difference in food intake was observed in these groups (FIG. 6).

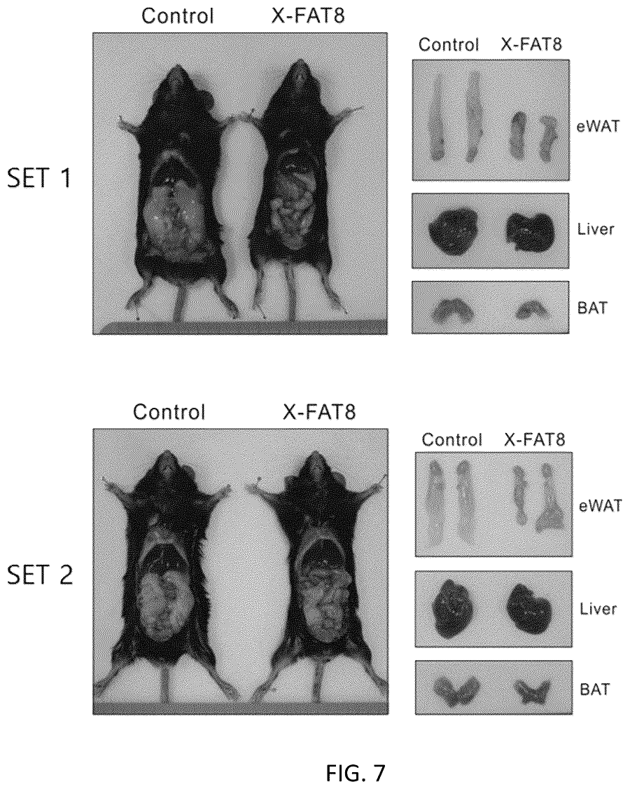

Meanwhile, for the X-FAT8-administered group, the mice were euthanized after completion of administration, and the state of organs and visceral fat were observed. As a result, it was confirmed that no special organ abnormalities were detected and the size of epididymal white adipose tissue (eWAT) significantly decreased in the X-FAT8-administered group (FIG. 7).

### Glucose Homeostasis Tests: GTT (Glucose Tolerance Test) and ITT (Insulin Tolerance Test)

GTT (glucose tolerance test) and ITT (insulin tolerance test) were performed to investigate whether there were any changes in in vivo glucose homeostasis. For the glucose tolerance test, mice were fasted for 16 hours and then orally administered glucose (Sigma-G8769), diluted to 20% glucose, at a dose of 1.5 g/kg. After glucose infusion, blood was collected from the mouse tail at 0, 15, 30, 60, and 120 minutes and measured using a self-monitoring blood glucose meter. For the insulin tolerance test, mice were fasted for 6 hours and then administered an intraperitoneal injection of insulin (NovoRapid Inj., 100 units/mL) diluted to 0.75 U/kg insulin. After insulin injection, blood was collected from the mouse tail at 0, 15, 30, 60, and 120 minutes and measured using a self-monitoring blood glucose meter (Roche Diagnostics Korea Co., Ltd.).

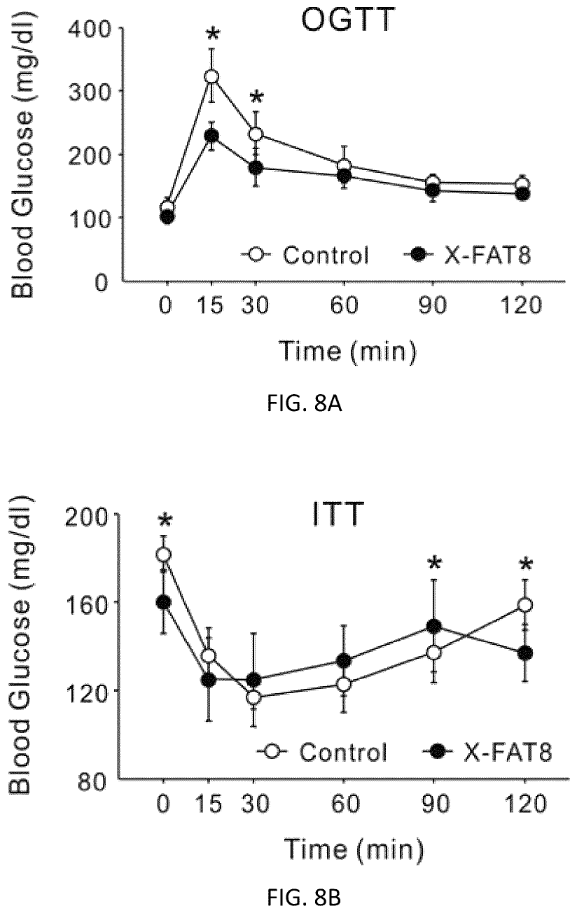

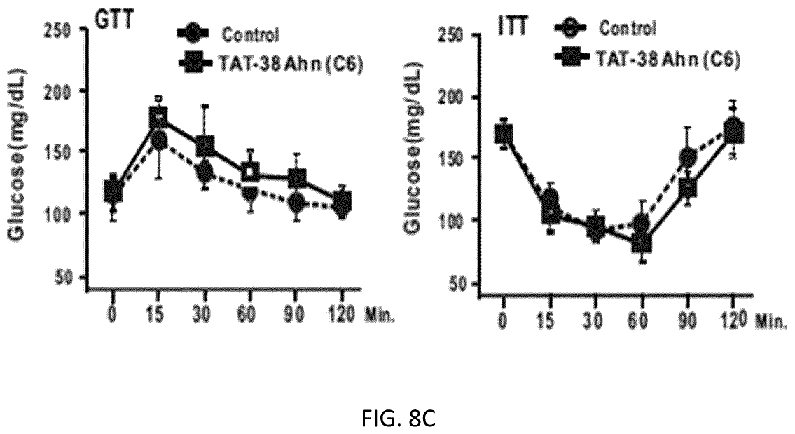

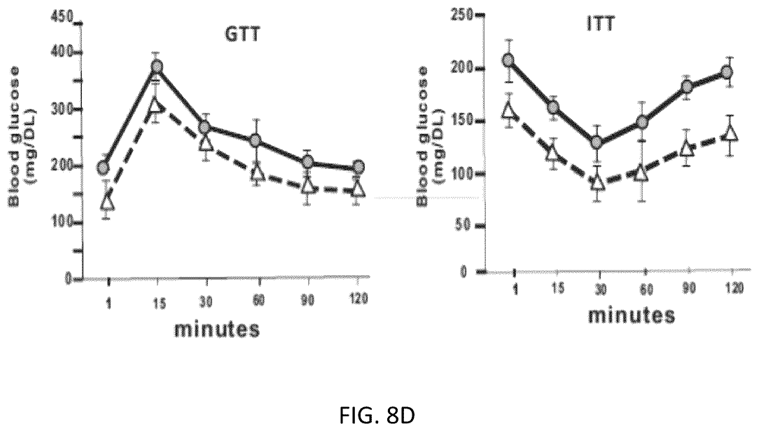

As a result of administering X-FAT8 (FIG. 8A) and X-FAT6 (FIG. 8B), it could be seen that the blood glucose concentration in the GTT results was either not different or lower and reached equilibrium more quickly than that in the control group, and there was no significant difference in insulin sensitivity. Thereby, it was confirmed that both X-FAT8 and X-FAT6 did not affect in vivo glucose uptake and homeostasis, suggesting that they can be used as a composition for treatment of various lipid metabolism abnormalities, including obesity.

### Histological Analysis of Liver Tissue and White Adipose Tissue

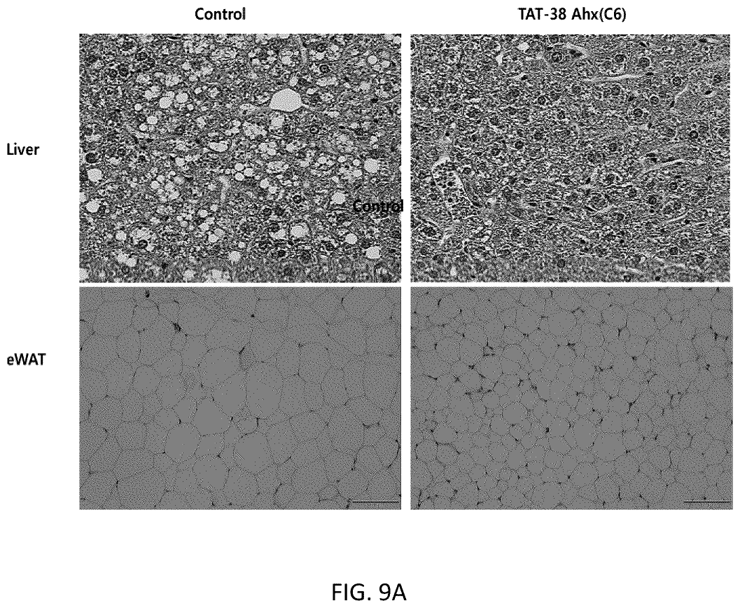

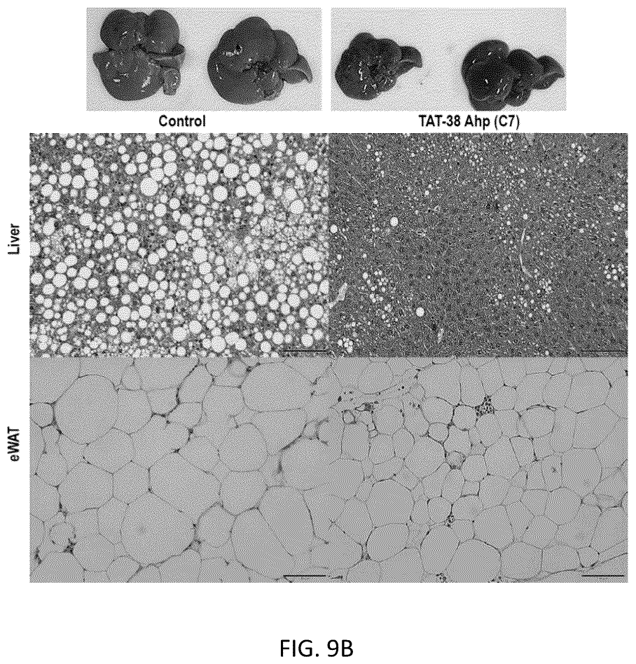

Tissues were harvested from the mice administered with vehicle (sample-dissolving solvent (sterile distilled water)) or X-FAT6 or X-FAT7, washed with PBS buffer, and then fixed with 10% neutral formalin overnight. Next, the adipose tissue and liver tissue were embedded in paraffin to make tissue paraffin blocks which were then sectioned with a microtome to obtain tissue slices. Staining the tissue slices with hematoxylin and eosin (H&E) reagents and microscopic analyses of the H&E stained tissues revealed that the size of the adipocytes in the epididymal white adipose tissue significantly decreased, and also the amount of stored fat in the liver tissue was significantly decreased, and the liver tissue was restored to cherry red healthy liver tissue (FIGS. 9A and 9B).

### Blood Biochemistry Test

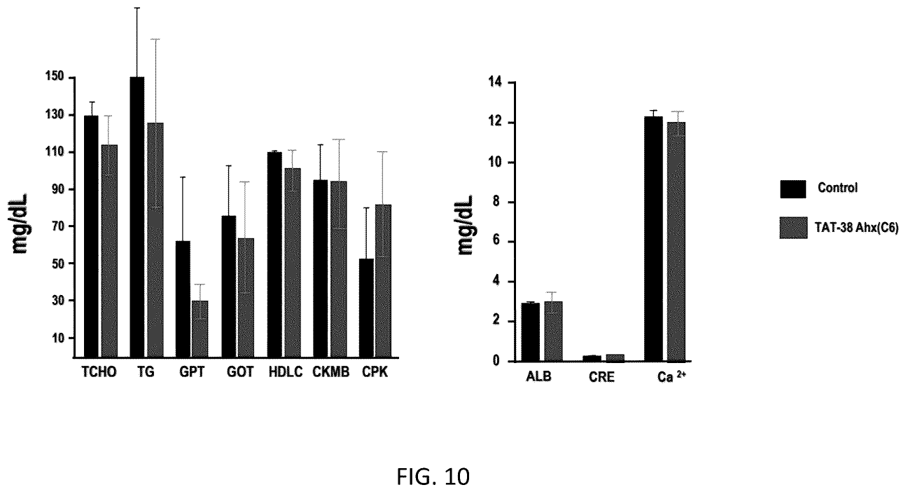

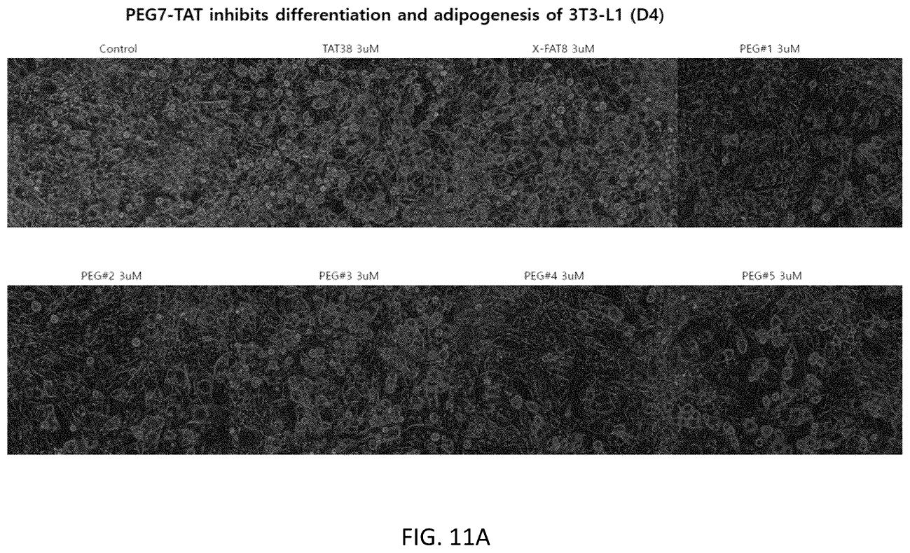

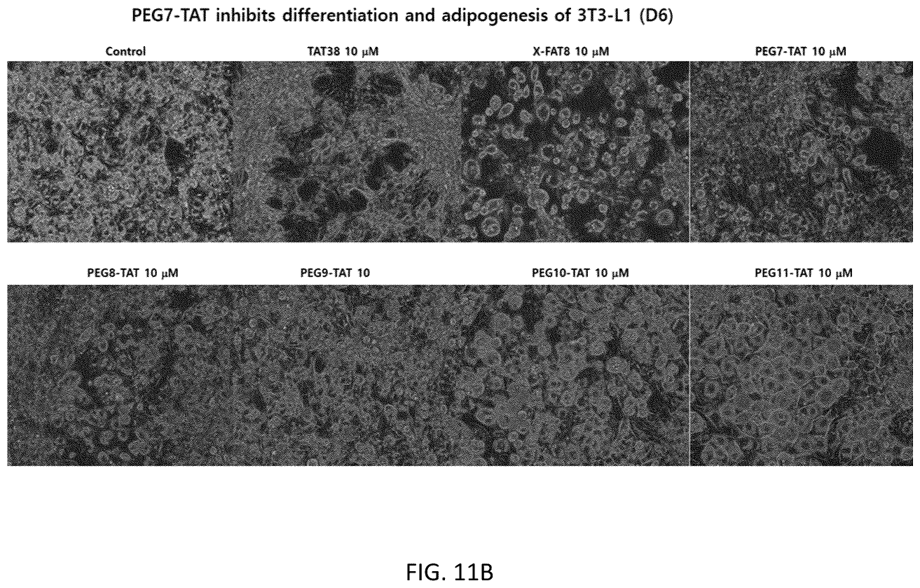

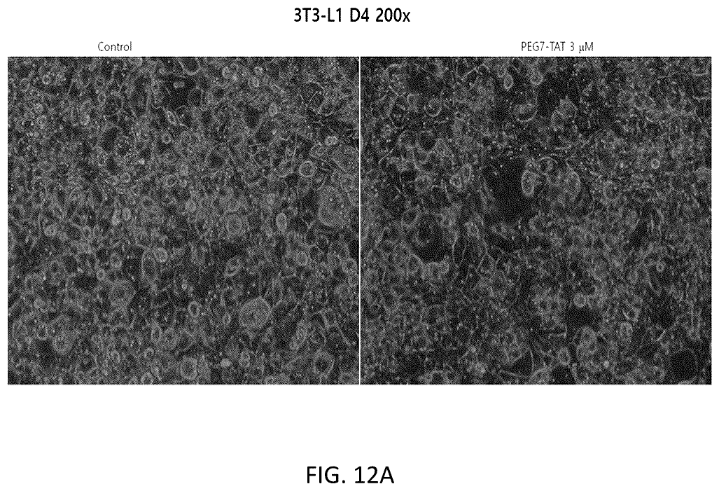

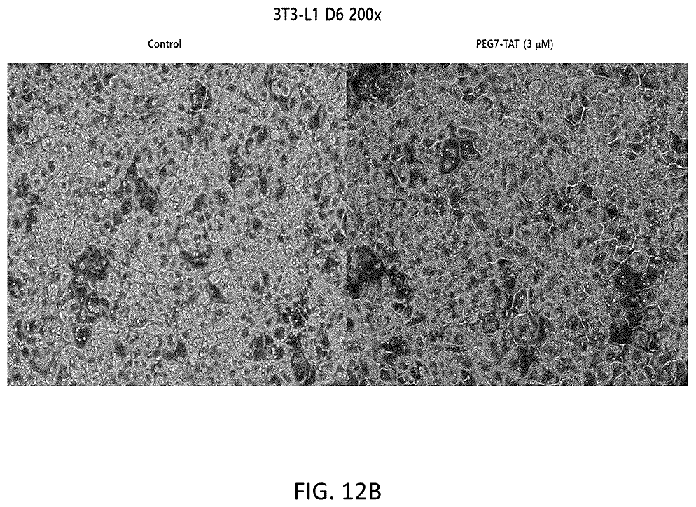

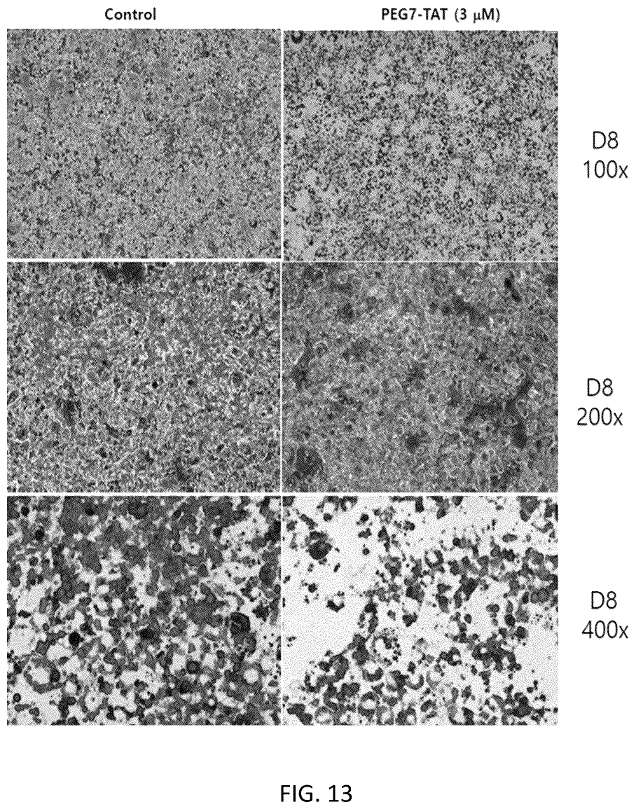

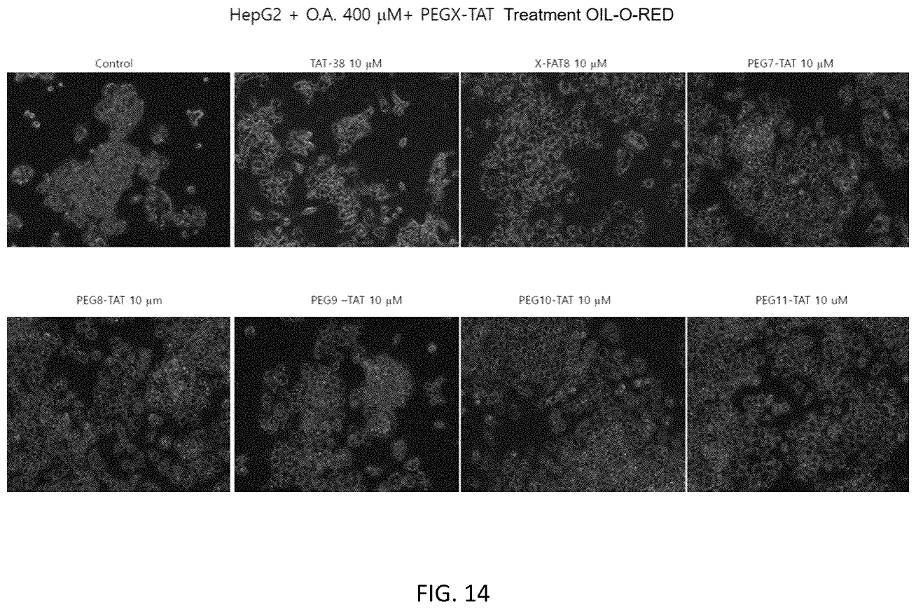

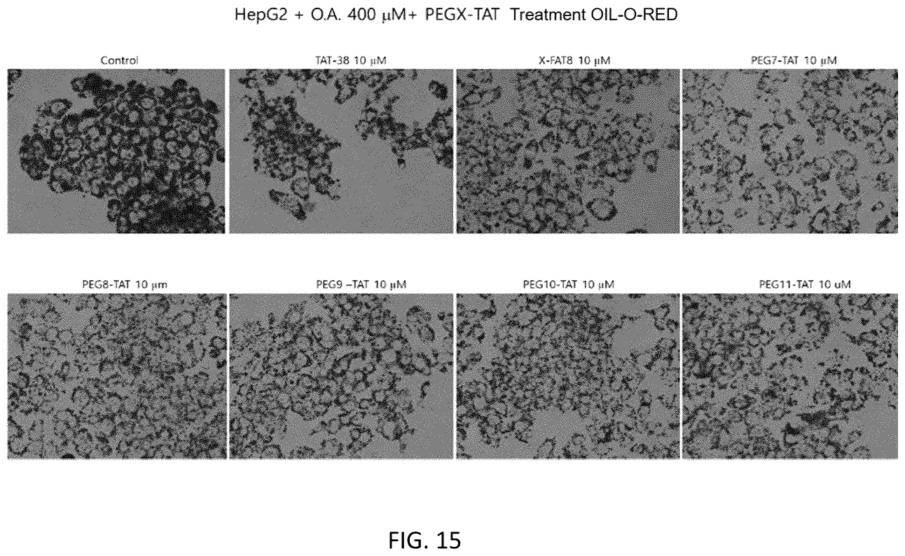

After completion of TA38 Ahx(C6) (X-FAT6) administration, the mice were euthanized, blood was collected, and serum was separated therefrom by centrifugation (15,000 rpm, 5 minutes, 4° C.). The levels of HDL-C (high-density lipoprotein cholesterol), TCHO (total cholesterol), TG (triglyceride), liver damage marker aminotransferase ALT (alanine aminotransferase), AST (aspartate transferase), Ca (calcium), ALB (albumin), CRE (creatinine), CK-MB (creatinine kinase MB), and CPK (creatine phosphokinase) in the separated serum were measured with a blood analyzer (FUJIFILM—DRI-CHEM NX500i) using FDC slides. These data suggested that administration of X-FAT6 significantly reduced blood triglyceride and cholesterol levels and improved liver function, by inhibiting adipocyte differentiation and fatty acid synthesis (FIG. 10).

### Example 2: PEGn-TAT-38 Peptide Variants

The present inventors further developed new TAT variants with improved drug properties and efficacy, based on information obtained through the discovery of TAT-38 and X-FATn variants.

As a method of improving the physical properties or efficacy of a drug, the process of conjugating linear or branched polyethylene glycol (PEG) molecules to the drug is called PEGylation. The most common form of polyethylene glycol (HO—(CH₂CH₂O)n-CH₂CH₂—OH) is a linear or branched polyether terminated with OH groups. The reason why PEGylation is advantageous for drug delivery is because it is excellent at sequestering and preserving the drug and has selectivity. In an aqueous solution, polyethylene glycol molecules are hydrated, move quickly, and occupy a large volume, preventing other molecules from approaching or interfering with the drug. Therefore, it is possible to protect the drug from immune response and clearance and increase drug bioavailability.

Accordingly, it was found that, when the core domain among the TAT peptide domains, which binds to the CDK9-Cyclin T1 complex and plays a key role in fat reduction, is replaced with PEG having a length similar to the core domain, the resulting TAT peptide variant has excellent pharmaceutical properties, including excellent stability and increased in vivo half-life, while retaining the fat reduction effect of the natural TAT peptide.

### Design and Synthesis of PEGNn-TAT Variants

TAT variants were synthesized by linking 20-37 a.a (TACTNCYCAKCCFHCQVC) and 49-57 a.a (RAKRRQRRR) peptides of the TAT peptide through PEGylation.

TAT-PEGylated Peptides
Cys-ZF-(Peg)n-PTD Peptide

TACTNCYCAKCCFHCQVC-(CH2CH2O)nCH2CH2-
RAKRRQRRR
TACTNCYCAKCCFHCQVC-(Peg)4-RAKRRQRRR
TACTNCYCAKCCFHCQVC-(Peg)5-RAKRRQRRR
TACTNCYCAKCCFHCQVC-(Peg)6-RAKRRQRRR
TACTNCYCAKCCFHCQVC-(Peg)7-RAKRRQRRR
TACTNCYCAKCCFHCQVC-(Peg)8-RAKRRQRRR
TACTNCYCAKCCFHCQVC-(Peg)9-RAKRRQRRR
TACTNCYCAKCCFHCQVC-(Peg)10-RAKRRQRRR
TACTNCYCAKCCFHCQVC-(Peg)11-RAKRRQRRR

Along with the peptides in which N-terminus and C-terminus of above peptides are modified by acetylation and amidation, respectively.

Through the design of PEGylated variants as shown in the above schematic diagram, the hydrophilicity of the TAT peptide was further increased, the stability of the TAT peptide against degrading enzymes was improved, and the pharmacological activity of the TAT peptide was dramatically increased due to an increase in half-life. The peptides were synthesized with a purity of 95% or more by Lifetein LLC (https://www.lifetein.com) located in New Jersey, USA.

### Analysis of Ability to Inhibit Differentiation of 3T3-L1 Cells and Inhibit Fatty Acid Synthesis

3T3-L1 cells were cultured in DMEM (high glucose, pyruvate, GIBCO CAT #11995) supplemented with 10% calf serum and 1% penicillin-streptomycin (P/S). Cells were seeded in a 6-well culture plate, and when the cells reached a confluency of 60 to 70%, MDI, a differentiation initiation medium, was added at a 1× concentration. At this time, the cells were treated with each of TAT-38, X-FAT8, and PEGn-TAT at a concentration of 1 to 10 μM. After 2 days, MDI was replaced with insulin, and each of AT-38, X-FAT8, and PEGn-TAT was added to fresh medium every day during culture. From day 4, culture was continued for 4 days in maintenance medium. Thereafter, the cell status was checked and recorded every day. When necessary, the cells were fixed and the amount of fatty acids was analyzed by Oil-O red staining.

### Effect on Fatty Acid Synthesis by Oleic Acid Stimulation in HEPG2 Hepatocytes

HEG2 cells were cultured in MEM (minimum essential medium eagle) (Welgene cat. LM 007-07) supplemented with 10% FBS and 1% P/S. When the cells reached a confluency of 60 to 70%, they were treated with oleic acid (500 nM) and the PEGylated TAT variant (10 μM), and then cultured for an additional 24 hours, and the amount of lipids in the cells was analyzed by Oil-O-Red staining.

TABLE 1
Sequence listing

| SEQ ID | |
|---|---|
| NO. | Amino acid sequence |
| 1 | MEPVNPRLEP WKHPGSQPKT ACTNCYCAKC CFHCQVC |
| 2 | RAKRRQRRRP PQGSQTHQVS LSKQ |
| 3 | TACTNCYCAK CCFHCQVC |
| 4 | RAKRRQRRR |
| 5 | MEPVNPRLEP WKHPGSQPKT ACTNCYCAKC CFHCQVCFIT |
| | KALGISYGRA KRRQRRRPPQ GSQTHQVSLS KQ |

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1. A compound represented by following Formula 1 or a pharmaceutically acceptable salt thereof: 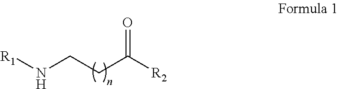
wherein R₁ is a polypeptide having the amino acid sequence of SEQ ID NO: 1 or a partial fragment of a C-terminal region thereof, R₂ is a polypeptide having the amino acid sequence of SEQ ID NO: 2 or a partial fragment of an N-terminal region thereof, and n is an integer of 3 to 7.

2. The compound or pharmaceutically acceptable salt thereof according to claim 1, wherein n is an integer of 3 to 6.

3. The compound or pharmaceutically acceptable salt thereof according to claim 2, wherein n is 4.

4. The compound or pharmaceutically acceptable salt thereof according to claim 1, wherein R₁ is the polypeptide having the amino acid sequence of SEQ ID NO: 1 or a fragment having 18 or more amino acid residues which are consecutive in a direction from the C-terminus to the N-terminus of SEQ ID NO: 1.

5. The compound or pharmaceutically acceptable salt thereof according to claim 4, wherein the fragment is a polypeptide having the amino acid sequence of SEQ ID NO: 3.

6. The compound or pharmaceutically acceptable salt thereof according to claim 1, wherein R₂ is the polypeptide having the amino acid sequence of SEQ ID NO: 2 or a fragment having 9 or more amino acid residues which are consecutive in a direction from the N-terminus to the C-terminus of SEQ ID NO: 2.

7. The compound or pharmaceutically acceptable salt thereof according to claim 6, wherein the fragment is a polypeptide having the amino acid sequence of SEQ ID NO: 4.

8. A pharmaceutical composition for preventing or treating a metabolic disorder selected from the group consisting of obesity, diabetes, dyslipidemia, fatty liver, and insulin resistance syndrome, comprising the compound or pharmaceutically acceptable salt thereof according to any one of claims 1 to 7 as an active ingredient.

9. The composition according to claim 8, wherein the dyslipidemia is hyperlipidemia.

10. The composition according to claim 8, wherein the fatty liver is non-alcoholic fatty liver.

11. A food composition for ameliorating or alleviating a metabolic disorder selected from the group consisting of obesity, diabetes, dyslipidemia, fatty liver, and insulin resistance syndrome, comprising the compound or pharmaceutically acceptable salt thereof according to any one of claims 1 to 7 as an active ingredient.

12. A compound represented by following Formula 2 or a pharmaceutically acceptable salt thereof: 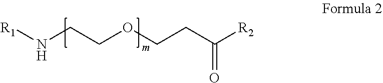
wherein R₁ is a polypeptide having the amino acid sequence of SEQ ID NO: 1 or a partial fragment of a C-terminal region thereof, R₂ is a polypeptide having the amino acid sequence of SEQ ID NO: 2 or a partial fragment of an N-terminal region thereof, and m is an integer of 4 to 7.

13. The compound or pharmaceutically acceptable salt thereof according to claim 12, wherein m is an integer of 6 to 8.

14. The compound or pharmaceutically acceptable salt thereof according to claim 13, wherein m is 7.

15. The compound or pharmaceutically acceptable salt thereof according to claim 12, wherein R₁ is the polypeptide having the amino acid sequence of SEQ ID NO: 1 or a fragment having 18 or more amino acid residues which are consecutive in a direction from the C-terminus to the N-terminus of SEQ ID NO: 1.

16. The compound or pharmaceutically acceptable salt thereof according to claim 15, wherein the fragment is a polypeptide having the amino acid sequence of SEQ ID NO: 3.

17. The compound or pharmaceutically acceptable salt thereof according to claim 12, wherein R₂ is the polypeptide having the amino acid sequence of SEQ ID NO: 2 or a fragment having 9 or more amino acid residues which are consecutive in a direction from the N-terminus to the C-terminus of SEQ ID NO: 2.

18. The compound or pharmaceutically acceptable salt thereof according to claim 17, wherein the fragment is a polypeptide having the amino acid sequence of SEQ ID NO: 4.

19. A pharmaceutical composition for preventing or treating a metabolic disorder selected from the group consisting of obesity, diabetes, dyslipidemia, fatty liver, and insulin resistance syndrome, comprising the compound or pharmaceutically acceptable salt thereof according to any one of claims 12 to 18 as an active ingredient.

20. A food composition for ameliorating or alleviating a metabolic disorder selected from the group consisting of obesity, diabetes, dyslipidemia, fatty liver, and insulin resistance syndrome, comprising the compound or pharmaceutically acceptable salt thereof according to any one of claims 12 to 18 as an active ingredient.
